(19) United States
(12) Patent Application Publication
(10) Pub. No.: US 2025/0355280 A1
(43) Pub. Date: Nov. 20, 2025

(54) BINOCULAR LOUPES AND METHODS OF MANUFACTURING BINOCULAR LOUPES

(71) Applicants: Kikuko YONEZAWA, Higashichikuma-gun, Nagano (JP); MEDSOLEIL CO., LTD., Higashichikuma-gun, Nagano (JP)

(72) Inventor: Kikuko YONEZAWA, Higashichikuma-gun, Nagano (JP)

(21) Appl. No.: 18/714,197

(22) PCT Filed: Sep. 21, 2023
(86) PCT No.: PCT/JP2023/034190

§ 371 Date: Oct. 24, 2024

(30) Foreign Application Priority Data
Sep. 26, 2022 (JP) 2022-152236
Mar. 20, 2023 (JP) 2023-043796

(51) Int. Cl. G02C 7/08

(52) U.S. Cl. CPC G02C 7/088; G02C 7/081

(57) ABSTRACT

Provided are methods of manufacturing binocular loupes which enable loupe bodies including desired focus adjustment units and magnifications to be replaced, and which have bright clear image quality while more widening the field of view of a practitioner, the binocular loupes are provided with a pair of right-and-left loupe bodies 3 with internal optical systems, a glasses frame 10 to hold carrier lenses 4 for supporting the pair of loupe bodies 3 in visual directions toward an observation target, and cylindrical loupe holders 9 attached to the carrier lenses 4 to receive the loupe bodies 3 attachably/detachably with eyepiece side ends of the pair of loupe bodies 3 inserted, and the loupe holders 9 and at least eyepiece side members of the pair of loupe bodies 3 are formed to be thin and high in magnetic flux density performance, by methods of manufacturing plastic magnet materials molded by mixing magnetic powder into plastic materials by precision Europe high compression molding, multi-gate injection molding or the like.

### TECHNICAL FIELD

The present invention relates to binocular loupes which are used in hand operation with high accuracy in surgical operations and precision processing, and which enlarge a visual identification target to observe, while allowing vision correction of users, methods of manufacturing the binocular loupes, and particularly, binocular loupes for medical practitioners.

### BACKGROUND ART

Binocular loupes have conventionally been used widely in various fields such as medical fields, precision work and jewel processing, as means for enlarging a fine local observation target on hand to visually identify. In these fields, high accuracy is required in operation of fine work on hand, and it is important that the binocular loupes provide, to practitioners, images of bright clear high image quality, in addition to excellent resolution, wide field of vision, focal distance and the like.

Particularly, in binocular loupes used in the medical field, the loupes are involved in human health and life, and therefore, are required to have functions such as proper vision correction and astigmatism correction by the binocular loupes in accordance with vision of the practitioner.

However, in conventional binocular loupes, regardless of high accuracy required in handwork of medical practitioners, with respect to vision cocreation to nearsightedness or farsightedness of the practitioner, and adjustments to focal distances of lenses, since it is not possible to adjust eyesight of the practitioner during an operation, while adapting to the type of the operation or situations changing during the operation, there is the problem that variations occur in accuracy of visual field and vision.

Further, the vision of a human varies every moment corresponding to the extent of physical conditions and fatigue, and even in the case where the same user uses, the vision and the like often vary in the morning and afternoon on the same day. Thus, it is not possible to adapt the conventional binocular loupes to vision varying every moment of the practitioner, and the practitioner has often performed operation work appropriately selecting binocular loupes in improper vision states to use.

Therefore, conventionally, binocular loupes have been known where a plurality of types of focus adjustment units with different focal distances is beforehand prepared, and one of the units is selected to enable the unit to be installed in an eyepiece unit thereof attachably/detachably (for example, see Patent Document 1).

FIG. 18 illustrates the type of binocular loupes described in Patent Document 1, and shows an example of first conventional technique where a loupe body is fixedly inserted into a carrier lens.

In FIG. 18, a loupe body 3 is provided with an internal enlargement optical system to adjust an image of an observation target under a predetermined magnification, for example, such as a two-time magnification and three-time magnification, and is comprised of an eyepiece barrel positioned in an end on the eyepiece side, and through a tilting portion for gradually increasing an outside diameter therefrom, a barrel in which is disposed an objective lens provided with a large-diameter portion.

Herein, further, when the loupe body 3 is provided inside with a zoom mechanism for enabling a mutual distance between an eyepiece-side lens group positioned in the end on the eyepiece side, and an objective-side lens group to be variable, it is possible to vary a focal distance continuously.

Then, into a rear end of the eyepiece barrel is inserted a magnetic ring 6 that sticks to a magnet. Further, on the eyepiece end side of the loupe body 3, a magnetic lens holding unit 12 is made along a circumference of a focus adjustment lens 11 so that the circular focus adjustment lens 11 is fitted. In order to correct the eyesight when a user of the binocular loupes enlarges and observes the target by right and left loupe bodies 3, this focus adjustment lens 11 is capable of being also made a lens to correct astigmatism and the like, not only to correct a far distance or a near distance used corresponding to the loupe body 3 as necessary.

Therefore, a focus adjustment ring (not shown) to adjust height of focus adjustment may be provided inside the focus adjustment lens 11. Further, even when a user of the binocular loupes does usually not need correction of the eyesight, adjustments to the eyesight are ensured, by using the focus adjustment lens 11 according to variations in the eyesight (nearsightedness or farsightedness) of the practitioner during treatment.

Thus, in the example of the conventional technique shown in FIG. 18 where the loupe body is fixedly inserted into the carrier lens, although the focus adjustment lens 11 is configured to be attachable and detachable with respect to the eyepiece unit end side of the loupe body 3, since the loupe body 3 is attached fixedly to the carrier lens 4, in the case of varying the magnification, it is necessary to replace the binocular loupes themselves including a glasses frame with other binocular loupes.

On the other hand, in the binocular loupes, since a required magnification differs corresponding to a part to treat, types of binocular loupes are also known where a plurality of kinds of binocular loupes is prepared, and as occasion demands, binocular loupes of an optimum magnification are selected to wear (for example, see Patent Document 2).

FIG. 19 illustrates the type of the binocular loupes described in, for example, Patent Document 2, and shows an example of second conventional technique of the type where a loupe body to use is selected as necessary from among a plurality of kinds of binocular loupe bodies beforehand prepared and is inserted into the carrier lens attachably/detachably.

In FIG. 19, it is configured that a loupe holder 9 is attached to enable the loupe body 3 to be received in the carrier lens 4 attachably/detachably. As shown in FIG. 19, it is configured that the loupe holder 9 is attached to the carrier lens 4 fitted into a glasses frame 10, and that one loupe body 3 selected from among a plurality of kinds of loupe bodies beforehand prepared is attached to the loupe holder 9 attachably/detachably.

Then, in the example shown in FIG. 19, the loupe body 3 is attached attachably/detachably to the loupe holder 9 fixed to the carrier lens 4, through a ring 6, and a magnet ring 5 provided with a plurality of concave portions in a circumference of a side of the loupe body. Then, in this example, in view of easiness in manufacturing and so on, the magnet ring 5 is comprised of two semicircular rings 5a, 5b that couple to be one by magnetic properties, and is engaged in the loupe holder 9 via the ring 6.

On the other hand, in the circumference of the loupe body 3 on the eyepiece side, convex magnetic protrusions 7 are provided to engage in the concave portions provided in the magnet ring 5, and the loupe body 3 is formed to be attracted to the magnet ring 5 so as to engage each other.

However, in the second conventional example, since the magnet ring 5 and magnetic ring 6 having relatively wide plane ring sides are connected to the circumference side on the eyepiece end side of the loupe body 3, the lens diameter for vision adjustment attached to the eyepiece end side of the binocular loupe is needed to be a small diameter. As a result, the field of view (vision) of the binocular loupe is narrowed, while the lens diameter is small, thereby interfering with making the optical system of the binocular loupe brighter. Particularly, as shown in FIG. 19, in order to reliably lock the loupe body 3, which is easy to install or remove, in the loupe holder by magnetic attraction forces, it is necessary to increase a volume (diameter×thickness) of each of the magnet ring 5 and magnetic ring 6.

Thus, in the second conventional example, as described above, a plurality of kinds of focus adjustment units with different focal distances is beforehand prepared to enable one to be selected from among the units and be inserted attachably/detachably. Further, in the conventional binocular loupes where a plurality of binocular loupes allowing the magnification to be adjusted is prepared to select optimal loupes as appropriate from among the loupes, since the structure of the binocular loupe body and the attachment unit (eyepiece unit) of the loupe holder for supporting the body is complicated, and parts of the attachable/detachable mechanism are high in number, the glass diameter of the ocular lens is thereby decreased, and as a result, the field of view (range of vision) of the practitioner is narrowed.

### PRIOR ART DOCUMENT

### Patent Document

Patent Document 1: Japanese Patent Gazette No. 5032332
Patent Document 2: Japanese Unexamined Patent Publication No. 2019-144297

### DISCLOSURE OF INVENTION

### Problems to be Solved by the Invention

The present invention was made in view of above-mentioned problems, and in binocular loupes enabling loupe bodies provided with internal optical systems for enlarging an observation target to visually identify, and focus adjustment units to be replaced easily with a desired magnification, it is an object of the invention to provide binocular loupes of bright and clear high image quality, while widening the field of view of a practitioner, and further, to provide specific methods of manufacturing the binocular loupes.

### Means for Solving the Problem

In order to solve the above-mentioned problem, the present invention is to provide binocular loupes for medical practitioners characterized in that the binocular loupes are provided with a pair of right-and-left loupe bodies with internal optical lens systems for enlarging an observation target on hand, right-and-left carrier lenses to support the pair of loupe bodies in visual directions toward the observation target, a glasses frame to hold the carrier lenses, and a pair of cylindrical loupe holders to attach the pair of loupe bodies respectively to the right-and-left carrier lenses attachably/detachably, the cylindrical loupe holders and contact portions contacting cylindrical inner surfaces of the loupe holders in the loupe bodies are formed of a plastic magnet member formed by mixing or kneading predetermined plastic materials with predetermined magnetic powder, the pair of loupe bodies are respectively locked closely in states of being attracted in the cylindrical inner surfaces of the loupe holders by magnetic attraction forces with the contact portions of the loupe bodies in the pair of loupe holders, and that a lubricant is applied to contact surfaces hitting cylindrical front end surfaces of the loupe holders to come into contact and eyepiece end side surfaces of the loupe bodies to improve slidability therebetween.

Thus, in the binocular loupes according to the present invention, the cylindrical loupe holder and at least the contact portion contacting the cylindrical inner surface of the loupe holder in the loupe body are formed of the plastic magnet member formed by mixing or kneading plastic materials with magnet powder, the loupe body is thereby locked closely in the cylindrical inner surface of the loupe holder by magnetic attraction forces without being dropped from the loupe holder, and as described later, it is made possible that the practitioner selects arbitrary one to insert and replace with ease from among the plurality of loupe bodies.

Herein, the loupe body is inserted into the loupe holder further through a protrusion-provided ring having a protrusion portion, the loupe holder has an engagement portion of a hook-shaped groove to engage in the protrusion portion, and it is thereby formed that the loupe body is not removed from the loupe holder.

Then, in the loupe body, a plurality of kinds of loupe bodies is beforehand prepared where the loupe bodies are provided with zoom mechanisms capable of varying a focal distance continuously in a single magnification with different focal distances or in a predetermined range, and one is selected from among the bodies to attach to the loupe holder. Further, as necessary, one of beforehand prepared vision adjustment lenses for far-sightedness and near-sightedness and astigmatism correction lenses is selected and fitted into the eyepiece end side surface of the loupe body attachably/detachably.

Further, as described later, the vision adjustment lens or astigmatism correction lens is held by a rim surrounding the circumference of the lens, and the rim is formed of a ferrite magnetic body, and is prepared and magnetized by the same method as an injection molding method of the loupe body described above.

In the loupe body provided with the zoom mechanism, the number of lenses increases, parts of the zoom mechanism are required, the diameter of a barrel portion of the loupe body is increased, and therefore, by forming of the above-mentioned plastic magnet member formed by mixing or kneading plastic materials with magnetic powder, it is possible to form thinner barrel portions of the loupe body.

Then, with respect to the plastic magnet member forming the cylindrical loupe holder constituting the binocular loupes, and at least the contact portion contacting the cylindrical inner surface of the loupe holder in the loupe body, as a first manufacturing method, the member is prepared by each of the following processes a) to e). In other words, the processes include that
a) the magnetic powder and thermosetting resin materials are mixed uniformly in an atmosphere of an inert gas,
b) the mixed raw material is subjected to press molding with a compression molder,
c) a shaped product subjected to the press molding is cured in a bonding furnace by heating,
d) the cured shaped product is subjected to barrel cleaning, surface treatment, and then, rust prevention treatment, and that
e) the external magnetic field is applied to the shaped product to magnetize.

Further, with respect to the plastic magnet member forming the cylindrical loupe body constituting the binocular loupes, and at least the contact portion contacting the cylindrical inner surface of the loupe holder in the loupe body, as a second manufacturing method, the member is prepared by each of the following processes a) to e). In other words, the processes include that
a) the magnetic powder and thermoplastic resin materials are charged into a kneader and are uniformly kneaded,
b) the kneaded raw material is pelletized to facilitate injection molding,
c) the pelletized raw material is subjected to dry treatment,
d) the dry-treated pelletized raw material is melted, and the melted raw material is subjected to injection molding into a desired shape die with an injection molder, and that
e) the external magnetic field is applied to the injection-molded shaped product to magnetize.

Herein, in process (d-1) of “injection molding” of the process d) in the second manufacturing method, the injection molder injects the melted raw material from one or a plurality of pinpoint injection gates into the shape die.

Further, in process (d-2) of “injection molding” of the process d) in the second manufacturing method, the injection molder injects the melted raw material into the shape die, using one or a plurality of side injection gates.

Furthermore, in process (d-3) of “injection molding” of the process d) in the second manufacturing method, the injection molder is also capable of injecting the melted raw material into the shape die, using a disk injection gate.

In addition, in the binocular loupes according to the present invention, in response to the extent of astigmatism and the like of the practitioner, it is necessary to adjust insertion angles of the loupe body in the loupe holder to predetermined angles.

Therefore, in order for each of the pair of loupe holders to be inserted in a predetermined rotation direction with respect to the loupe holder, at least each of the loupe holder and loupe body is formed of anisotropic magnetized materials where crystal molecular arrangements of the magnetic powder are oriented in a predetermined direction.

Thus, in the method of manufacturing the binocular loupes supporting astigmatism, with respect to the plastic magnet member forming the cylindrical loupe body, and at least the contact portion contacting the cylindrical inner surface of the loupe holder in the loupe body, as a third manufacturing method, the member is prepared by each of the following processes a) to f). In other words, the processes include that
a) the magnetic powder and thermoplastic resin materials are charged into a kneader and are uniformly kneaded,
b) the kneaded raw material is pelletized to facilitate injection molding,
c) the pelletized raw material is subjected to dry treatment,
d) the dry-treated pelletized raw material is melted,
e) the melted raw material is subjected to injection molding into a desired shape die in the magnetic field with an injection molder, and is demagnetized as necessary, and that
f) the external magnetic field is applied to the injection-molded shaped product to magnetize so that the crystal molecular arrangement of the magnetic powder is oriented in the predetermined direction.

In addition, as a first example of a structural member, the plastic magnet member is a structural member obtained by mixing or kneading samarium-iron-nitrogen (SmFeN)-based magnetic powder with predetermined plastic materials.

Then, as a second example of the structural member, the plastic magnet member is a structural member obtained by mixing or kneading samarium-iron-nitrogen (SmFeN)-based magnetic powder containing a ferrite iron oxide with predetermined plastic materials.

Further, as a third structural member, the plastic magnet member is a structural member obtained by mixing or kneading neodymium-iron-boron (NdFeB)-based magnetic powder with predetermined plastic materials.

In addition, methods of manufacturing the binocular loupes include a process of fixing the loupe holder at a predetermined angle with respect to the surface pf the loupe holder, so that the inserted loupe body is directed toward an observation target, and further, another process of fixing the loupe holder at another predetermined angle with respect to the surface pf the loupe holder, so that the inserted loupe body is directed toward the observation target.

Then, in the binocular loupes according to the present invention, in order that the focus adjustment lens is configured to be attachable and detachable with respect to the eyepiece unit end side with respect to the loupe body, and that a loupe body of an optimum magnification is attachable and detachable to the loupe holder corresponding to a part to treat, in the present invention, a plurality of kinds of loupe bodies is beforehand prepared where the loupe bodies are provided with zoom mechanisms capable of varying a focal distance continuously in a single magnification with different focal distances or in a predetermined range, and one is selected from among the bodies to attach to the loupe holder.

Further, as necessary, one of beforehand prepared vision adjustment lenses for far-sightedness and near-sightedness and astigmatism correction lenses is selected and fitted into the eyepiece end side surface of the loupe body attachably/detachably to use.

Herein, the vision adjustment lens or astigmatism correction lens is held by a rim surrounding the circumference of the lens, and the rim is formed of a ferrite magnetic body, and is prepared and magnetized by the same method as the injection molding method of the loupe body described above.

Further, in the present invention, in the loupe body provided with the zoom mechanism selectively attached to loupe holder, not only the contact portion contacting the cylindrical inner surface of the loupe holder, but also the barrel body with the internal zoom mechanism portion is prepared and magnetized by the same method as the injection molding method of the loupe body as described above. By this means, the operability of zoom operation is improved, while thinning the barrel body of the loupe body.

Then, the rim uses the samarium-iron-nitrogen (SmFeN)-based magnetic powder or the structural member obtained by mixing or kneading the samarium-iron-nitrogen (SmFeN)-based magnetic powder with predetermined plastic materials.

### Effect of the Invention

By this means, in the present invention, in the binocular loupes enabling loupe bodies with internal optical systems for enlarging an observation target to visually identify, and focus adjustment units to be easily replaced with a desired magnification, the invention specifically provides the method of manufacturing the binocular loupes provided with brighter and clear image quality, while further widening the field of view of a practitioner, as compared with the binocular loupes of conventional techniques.

### BRIEF DESCRIPTION OF DRAWINGS

FIG. 1 shows an example of an entire outer view of binocular loupes of the present invention;

FIGS. 2A and 2B illustrate explanatory views in inserting one of loupe bodies constituting the binocular loupes into a loupe holder on the carrier lens side, where FIG. 2A illustrates an explanatory view in the case of inserting the loupe body directly into the loupe holder, and FIG. 2B illustrates an explanatory view in the case of inserting the loupe body into the loupe holder via a protrusion-provided ring;

FIGS. 3A-3C illustrate explanatory views in inserting one of the loupe bodies constituting the binocular loupes into the loupe holder on the carrier lens side in response to respective configurations of FIGS. 2A and 2B, where FIG. 3A illustrates an explanatory view in the case of inserting the loupe body directly into the loupe holder, FIG. 3B illustrates an explanatory view in the case of inserting the loupe body into the loupe holder via the protrusion-provided ring, and FIG. 3C shows an example where a window (notch) is provided to ensure the field of vision of a practitioner in the case that the practitioner identifies hands through the carrier lens;

FIG. 4 shows an example of the binocular loupes and loupe bodies with a plurality of magnifications and/or vision adjustment distances capable of being inserted into the carrier lens of the binocular loupes;

FIGS. 5A-5C show one example of a loupe body 3 in the binocular loupes, where FIG. 5A illustrates a side elevational view of the loupe body 3, FIG. 5B illustrates a front view of the loupe body 3 viewed from the objective lens side 3d side, and FIG. 5C illustrates a sectional view of the body 3;

FIG. 6 illustrates an explanatory view in a state in which a practitioner conducts work, while wearing the binocular loupes;

FIG. 7 illustrates an explanatory view of a downward insertion angle in attaching the loupes to the carrier lenses;

FIG. 8 illustrates an explanatory view about inward insertion angles p, q in attaching the loupes to the carrier lenses;

FIG. 9 illustrates process steps of a press molding method that is a first manufacturing method of the binocular loupes (cylindrical loupe holders and loupe bodies);

FIG. 10 illustrates process steps of an injection molding method that is a second manufacturing method of the binocular loupes (cylindrical loupe holders and loupe bodies);

FIG. 11 illustrates an injection example of melted raw materials (plastic·magnet) into a shape die using a plurality of (four) pinpoint injection gates that is one of injection molding methods, which is the second manufacturing method of cylindrical loupe holders and the like of the binocular loupes shown in FIG. 10;

FIG. 12 illustrates an injection example of melted raw materials (plastic·magnet) into the shape die using side injection gates that is another injection molding method, which is the second manufacturing method of cylindrical loupe holders and the like of the binocular loupes shown in FIG. 10;

FIGS. 13A and 13B illustrate other injection molding methods which are the second manufacturing method of the cylindrical loupe holders and the like of the binocular loupe bodies shown in FIG. 10, where FIG. 13A illustrates an injection example of melted raw materials (plastic·magnet) into a shape die using a guide side injection gate, and FIG. 13B illustrates an injection example of melted raw materials (plastic·magnet) into the shape die from a guide pin injection gate;

FIGS. 14A and 14B illustrate an injection example of melted raw materials (plastic·magnet) into a shape die using a disk injection gate that is still another injection molding method which is the second manufacturing method of the cylindrical loupe holders and the like of the binocular loupes shown in FIG. 10, where FIG. 14A illustrates a perspective view thereof, and FIG. 14B illustrates a perspective view thereof viewed from the lateral direction;

FIG. 15 illustrates processes of generating anisotropy in magnetic orientations of magnetic powder of the injection-molded shaped product in an injection molding method that is a third manufacturing method of the binocular loupes (cylindrical loupe holders and loupe bodies);

FIGS. 16A and 16B illustrate a product example obtained by mixing or kneading samarium-iron-nitrogen (SmFeN)-based magnetic powder with plastic materials, as a first example of the plastic magnet member used in preparation of the loupe holders and loupe bodies of the binocular loupes of the present invention, where FIG. 16A shows magnetic properties, and FIG. 16B shows physical mechanical properties;

FIGS. 17A and 17B illustrate a product example obtained by mixing or kneading neodymium-iron-boron (NdFeB)-based magnetic powder with plastic materials, as a second example of the plastic magnet member used in preparation of the loupe holders and loupe bodies of the binocular loupes of the present invention, where FIG. 17A shows magnetic properties, and FIG. 17B shows physical mechanical properties;

FIG. 18 illustrates an explanatory view of conventional techniques, and shows an example of the first conventional technique where the loupe body is fixedly inserted into the carrier lens; and

FIG. 19 illustrates an explanatory view of conventional techniques, and shows an example of the second conventional technique where the loupe body is inserted attachably/detachably into the loupe holder on the carrier lens side.

### MODE FOR CARRYING OUT THE INVENTION

Embodiments of binocular loupes according to the present invention will be described below with reference to drawings.

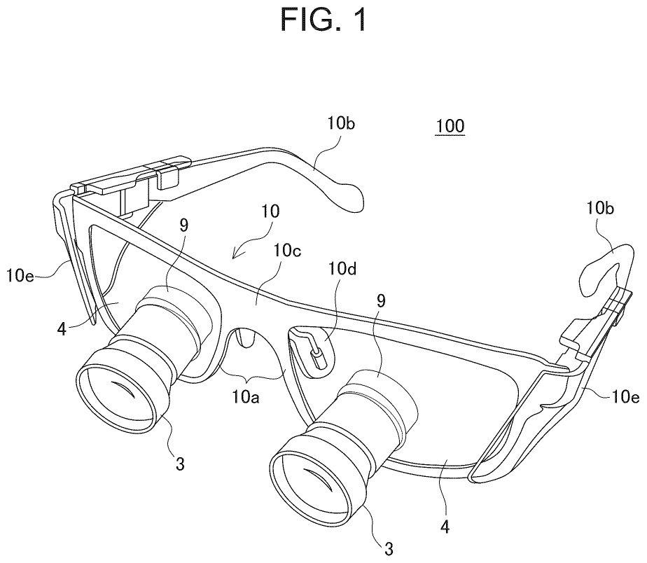

FIG. 1 illustrates an entire configuration of binocular loupes 100 according to one Embodiment of the present invention. The binocular loupes 100 are comprised of a glasses frame 10, loupe bodies 3 supporting right-and-left both eyes to enlarge an image of a work target, loupe holders 9 to hold the loupe bodies 3, and carrier lenses 4 that are loupe holders to attach the loupe holders 9 to the glasses frame 10.

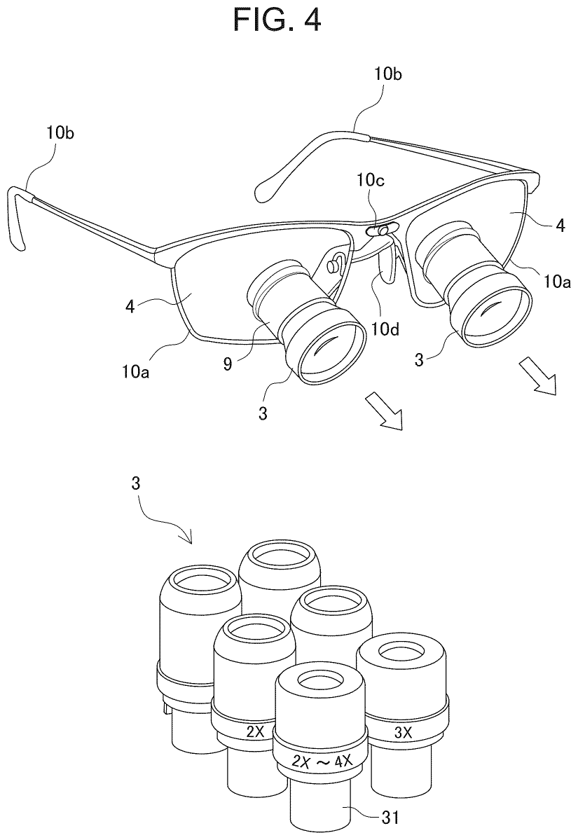

Herein, as shown in FIG. 4, there is a plurality of types of beforehand prepared loupe bodies 3 with different magnifications (e.g., three times, four times, five times) by optical systems and/or vision adjustment distances, and one of the bodies is selected and inserted into the loupe holder 9 attachably/detachably. In addition, as described later, with respect to vision adjustment functions, one of a plurality of beforehand prepared vision adjustment lenses or astigmatism correction lenses may be fitted attachably/detachably into an eyepiece side end of the loupe body 3. By this means, it is possible to decrease the types (the number) of beforehand prepared loupe bodies 3.

In FIGS. 1 and 4, the glasses frame 10 has substantially the same structure as normal glasses, and includes rims 10a into which the carrier lenses 4 are fitted, a bridge 10c for joining temples 10b put on ears of an observer and the rims 10a, and nose pad portions 10d. As materials constituting the glasses frame 10 are used metal such as titanium hard to rust with flexibility, synthetic resins and the like. Then, when necessary, it is possible to attach, to the temples 10b, shield members 10e to protect both sides of a face of a wearer, and straps (not shown) to keep the binocular loupes in a worn state.

In the carrier lenses 4, openings are pierced to support the loupe holders 9 for holding the loupe bodies 3 at opposite ends thereof, and the loupe holders 9 are fitted into the openings, and are fixed in states of maintaining predetermined angles with respect to surfaces of the carrier lenses 4. As materials constituting the carrier lenses 4, although the materials are not needed to be always transparent, the materials are preferably transparent to widen the field of vision of an observer in a hand direction. Further, correction lenses may be used in the case of needing correction of vision, and in the case without the need for correcting vision, simple transparent glasses may be used. In this case, materials of the lenses are glass or plastic. Accordingly, together with the function of the loupe holders for supporting the loupe bodies, as necessary, the carrier lenses 4 are also provided with the function of correcting vision.

In addition, in the loupe bodies 3 shown in FIG. 4, a loupe body shown by symbol 31 denotes a loupe body 3 enabling a practitioner to select a magnification of the lens and vision adjustment during treatment. Corresponding to the need during treatment, for example, a practitioner selects a loupe body 3 with a single focus such as three times (3×), four times (4×) and five times (5×) to insert into the loupe holder 9. Further, in the case where the loupe body 3 includes a zoom mechanism, by rotating or sliding back and forth a barrel on the objective lens size, the practitioner sets the magnification, for example, at 3 to 5 times (3-5×).

In addition, descriptions will be given later to details for fixing the loupe holder 9 in a state of maintaining the predetermined angle with respect to the surface of the carrier lens 4.

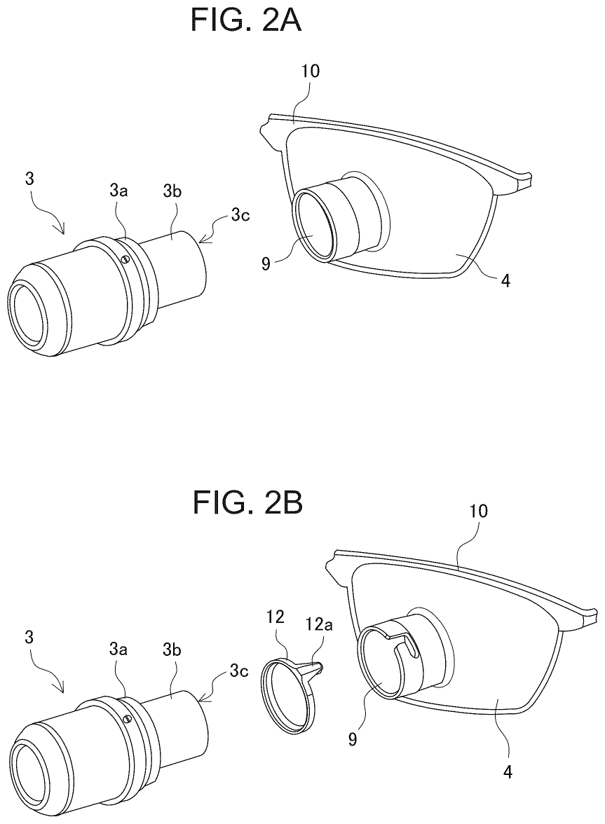

FIGS. 2A and 2B illustrate a state before the loupe body 3 is inserted into the loupe holder 9 fixed in the state of maintaining the predetermined angle with respect to the surface of the carrier lens 4, in the binocular loupes 100 according to the present invention shown in FIG. 1.

FIG. 2A illustrates the case where the loupe body 3 is directly inserted into the loupe holder 9, and then, FIG. 2B illustrates the case where the loupe body 3 is inserted into the loupe holder 9 with a protrusion-provided ring 12 nipped therebetween.

As shown in FIG. 2A, the loupe holder 9 is fixed to the carrier lens 4 at the predetermined angle, and the loupe body 3 is inserted into the loupe holder 9. In the loupe body 3, in a barrel body with a plurality of optical lenses stored are formed an intimate-contact surface 3b for contacting the inner surface of the cylindrical loupe holder 9 in an intimate-contact state, a contact surface 3a for hitting the cylindrical front end surface of the loupe holder 9 to come into contact therewith, and an eyepiece end side surface 3c of the loupe body 3.

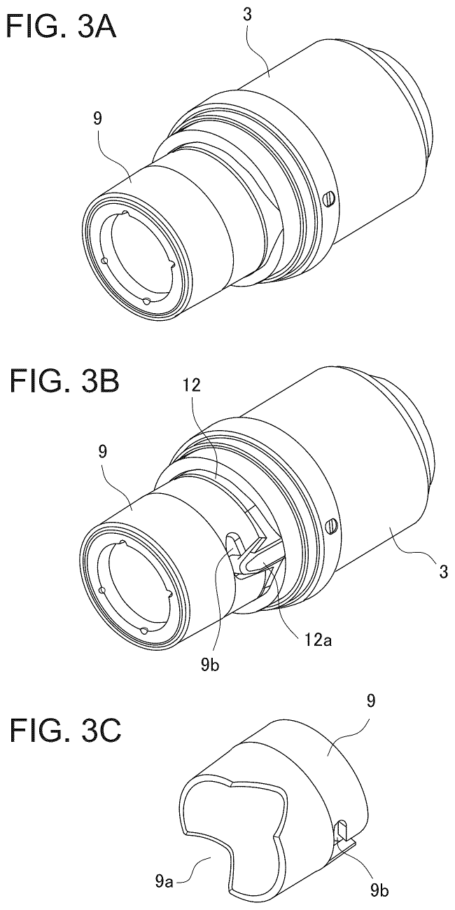

FIG. 2B illustrates the case where the loupe body 3 is inserted into the loupe holder 9 with the protrusion-provided ring 12 coming into intimate contact with the loupe body nipped therebetween. In this case, the loupe holder 9 includes an engagement portion 9b (see FIGS. 3A-3C) of a hook-shaped groove to engage in the protrusion portion 12a provided in the protrusion-provided ring 12, and by this means, for example, when a finger of the practitioner, lighting equipment of an operation room or the like contacts the loupe body 3, it is possible to prevent the loupe body 3 from being easily removed from the loupe holder 9.

In addition, the example shown in FIG. 2B illustrates the case where one protrusion portion 12a is provided in the protrusion-provided ring 12, and by providing a plurality of (for example, two or three) protrusion portions 12a, it is possible to more reinforce prevention of removal of the loupe body 3 from the loupe holder 9.

Further, in the example shown in FIG. 2A, as distinct from the example shown in FIG. 2B, the loupe body 3 is inserted directly into the loupe holder 9 attachably/detachably without passing through the protrusion-provided ring 12, and may be inserted into the loupe holder 9 attachably/detachably through a ring (not shown) without the protrusion portion 12a.

FIG. 3A illustrates a state in which the loupe body 3 shown in FIG. 2A is directly inserted into the loupe holder 9, and FIG. 3B illustrates a state in which the loupe body 3 shown in FIG. 2B is inserted into the loupe holder 9 with the protrusion-provided ring 12 nipped therebetween. As shown in FIG. 3B, the protrusion portion 12a of the protrusion-provided ring 12 engages in the engagement portion 9b of the hook-shaped groove provided on the loupe holder 9 side, and the loupe body 3 is thereby not easily removed from the loupe holder 9. In addition, the protrusion-provided ring 12 described above may be a ring 12 without the protrusion portion 12a.

Then, such a ring 12 may be formed of a plastic magnet described later, or may be formed of a magnetic metal ring magnetized in a predetermined direction.

Herein, with respect to the loupe holder 9, the inner contact surface 3b with the cylindrical loupe holder 9 on the eyepiece end side of the loupe body 3, the contact surface 3a for hitting the cylindrical front end surface of the loupe holder 9 to come into contact therewith, and the eyepiece end side surface 3c of the loupe body 3, the holder 9 and the surfaces 3a, 3b, 3c are formed of plastic magnets molded by mixing magnetic powder into plastic materials, or magnetic metal rings. By this means, the loupe body 3 is not removed from the loupe holder 9.

Herein, in order to enhance slidability between the contact surface 3a for hitting the cylindrical front end surface of the loupe holder 9 to come into contact therewith, and the eyepiece end side surface 3c of the loupe body 3, it is preferable to apply a lubricant (grease lubricant or oil lubricant) thin to therebetween. This is because a smooth slide is prevented in the case where a particle diameter is large in the magnetic powder to mix with plastic materials. Similarly, in the case where the above-mentioned ring 12a is formed of the plastic magnet, a lubricant may be applied to the above-mentioned contact surface 3a or the eyepiece end side surface 3c of the loupe body 3.

In addition, as shown in FIG. 3C, in the loupe holder 9, in order that the thickness of the loupe holder 9 does not interfere with the visual field of the carrier lens 4 when the practitioner views the hands through the carrier lens 4, not through the loupe body 3, for example, a window (notch) to ensure visibility may be provided in right-and-left two portions in the visual direction and/or on the lower side in the visual direction.

Further, when the loupe body 3 is inserted into the loupe holder 9 via the protrusion-provided ring 12 having the protrusion portion, by also forming the protrusion-provided ring 12 using the plastic magnet molded by mixing the magnetic powder into plastic materials, the loupe body 3 is promoted to maintain insertion into the loupe holder 9 by stronger magnetic forces.

Herein, it is preferable that at least the loupe holder 9 and all or a part of contact portions 3a, 3b, 3c with the loupe holder 9 in the loupe body 3 are formed of anisotropic magnetized materials where crystal molecular arrangements of the magnetic powder are oriented in a certain direction, and that the loupe body 3 is inserted into the loupe holder 9 in a predetermined rotation direction.

Further, in this case, by also forming the protrusion-provided ring 12 using the anisotropic magnetized materials where the crystal molecular arrangement of the magnetic powder is oriented in the certain direction, it is possible to more enhance the holding force of the loupe body in the loupe holder. As described above, it is possible to provide the protrusion portion 12a of the protrusion-provided ring 12 formed of the above-mentioned plastic magnet materials not only in a single portion, and also two portions or three portions.

Herein, the plastic magnet is molded by mixing magnetic powder into plastic, and it is possible to manufacture a magnetic plastic member in a desired shape using die sintering molding, press molding, injection molding, extrusion molding and the like. Further, by adding a small amount of rubber materials, it is also possible to manufacture members having elasticity.

With respect to strengths of magnetism, it is possible to manufacture magnetic compounds with various numeric values corresponding to magnetic materials to mix. For example, neodymium bonded materials (Nd—Fe—B) contain neodymium, iron and boron, are plastic magnets manufactured by combining and bonding resins thereto, enable press processing of thin shape and processing of complicated shape, are usually isotropy, and are capable of being magnetized in a diameter direction, height direction and multi-poles.

Thus, the plastic magnet is capable of being magnetized in a direction of magnetic field and strength of magnetic force in accordance with desires, and thereby enables the loupe holder 9 and all or a part of the contact portions 3a, 3b, 3c with the loupe holder 9 in the loupe body 3 in the binocular loupes 100 of the present invention to be magnetized in desired magnetic properties (N-pole, S-pole) and strengths of magnetic force.

From the aforementioned viewpoint, in the loupe holder 9 and loupe body 3 in the binocular loupes 100 of the present invention, for example, it is also possible to make one of the holder 9 and body 3 the plastic magnet of the above-mentioned neodymium bonded materials, while making the other one of the holder 9 and body 3 general ferrite materials or ferrite-based plastic magnet materials.

Anisotropic magnetization of the plastic magnet is one of techniques for varying properties and directions of magnetic force, as methods of aligning anisotropic easy axes of magnetization, there are axial anisotropy, radial anisotropy, repulsion radial anisotropy, polar anisotropy and the like, and the magnet is produced by orientating the magnetic field in a desired direction, for example, such as a vertical axis direction and horizontal axis direction, by the magnetic field of a magnet of an injection molding apparatus and inside a die in molding.

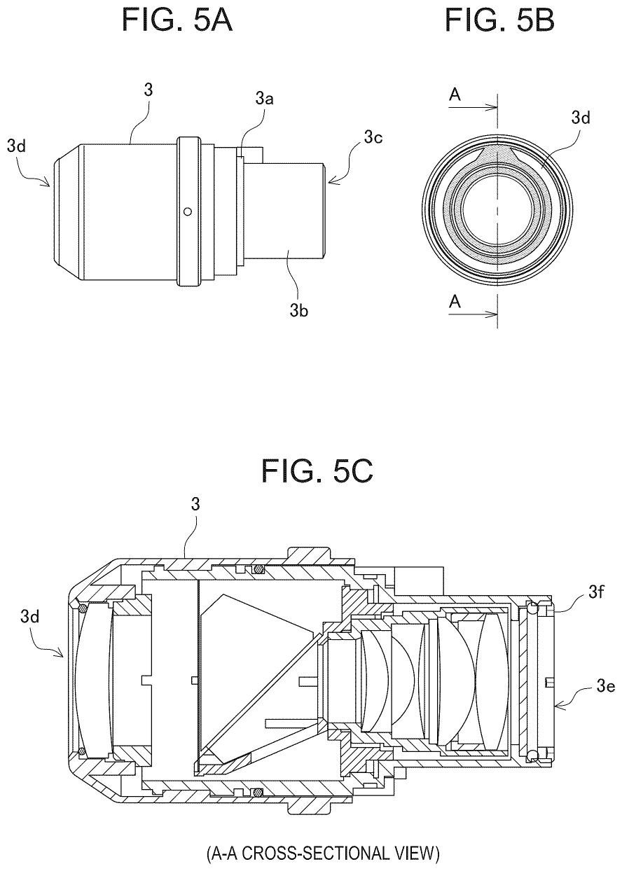

FIGS. 5A-5C show an example of the loupe body 3. FIG. 5A illustrates a side elevational view of the loupe body 3, FIG. 5B illustrates a front view viewed from the objective lens 3d side of the loupe body 3, and FIG. 5C illustrates a cross-sectional view.

As shown in FIG. 5C, the loupe body 3 is comprised of a plurality of optical lenses and a plurality of mechanism parts along an optical axis line from the objective lens 3d to the ocular lens 3e inside the cylindrical body.

In addition, the loupe body 3 constituting the binocular loupes according to the present invention includes not only a loupe of a single focus lens where the focal distance is fixed, and also a loupe body including a zoom mechanism capable of continuously enlarging a part of an observation target to view by varying the focus distance continuously by one body. In recent years, also in an F value (brightness) and image quality accuracy of a lens, it has been made possible to use a lens having a zoom mechanism equivalent to a single focus lens, and also in medical binocular loupes, it is possible to use a loupe body 3 provided with such a zoom mechanism. Also in the binocular loupes according to the present invention, by inserting the loupe body 3 provided with the zoom mechanism into the loupe holder 9, the single loupe body 3 is capable of enlarging a small observation part to view, and further, of zooming out the entire treatment part to view.

Herein, for example, the zoom mechanism is to continuously vary the focal distance by varying a mutual distance between a lens or a lens group on the eyepiece side (for example, a lens disposed inside symbol 3b portion shown in FIG. 5A) and a lens or a lens group on the objective side (a lens disposed inside a symbol 3 portion shown in FIG. 5A), in lens groups constituting the loupe body 3 shown in FIG. 5C. In order to vary the mutual distance between both lens groups, there are a rotation type for rotating a zoom ring (not shown) to vary the focal distance, and a straight type for moving a zoom ring back and forth to vary the focal distance, and any type is available as the loupe body 3.

As one example, by rotating the barrel on the objective lens side of the loupe body 3 shown in FIG. 4, the focal distance (magnification) is varied. Details of the zoom mechanism are publicly known to a person skilled in the art, and therefore, detailed descriptions thereof and movement of each part are omitted.

In addition, a plurality of beforehand prepared vision adjustment lenses 3f or astigmatism correction lenses may be fitted into the eyepiece side end of the loupe body 3 attachably/detachably. In this case, the vision adjustment lens 3f or astigmatism correction lens is preferably supported by a thin rim of a ferrite magnetic body surrounding the circumference of the lens.

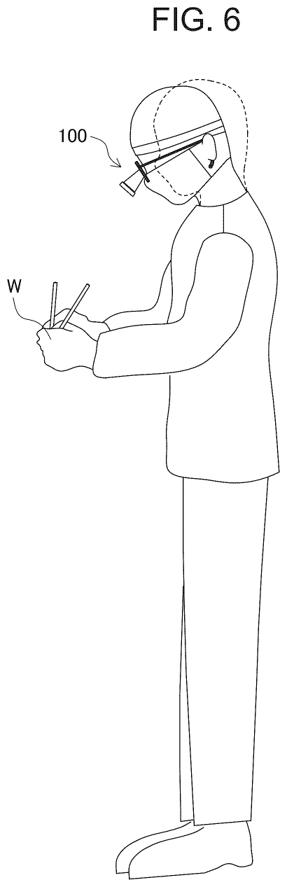

Next, based on FIGS. 6, 7 and 8, descriptions will be given to attachment of the loupe holder 9 to the surface of the carrier lens 4.

The loupe holder 9 is fitted into the opening pierced in the carrier lens 4, and is fixed at a predetermined angle with respect to the surface of the carrier lens 4, so that the loupe body 3 is directed toward a focus in an observation target direction. Specifically, attachment of the loupe holder 9 to the surface of the carrier lens 4 is determined by a downward insertion angle r and inward insertion angles p, q with reference to a plane of the carrier lens 4 of the loupe body 3 when the loupe body 3 is inserted into the loupe holder 9, and a distance PD between pupils of a user of the binocular loupes.

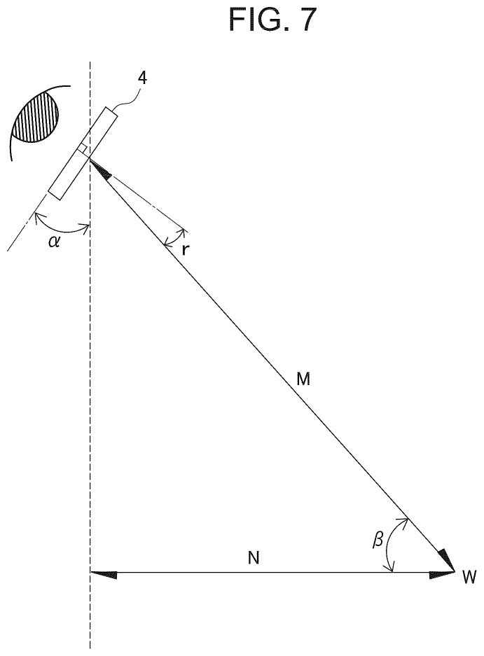

The binocular loupes 100 are used to enlarge an observation target in a work operation point W on hand to observe, in performing the work while leaning forward as shown in FIG. 6. At this point, the user concentrates lines of sight of both eyes on the observation target in a position of fingers through the right and left binocular loupes 3. Herein, using FIG. 7, the downward insertion angle will be described in this case. The downward insertion angle r is a downward attachment angle with respect to a perpendicular line from the surface of the carrier lens 4, in attaching the loupe body 3 to the carrier lens 4 through the loupe holder 9, and is capable of being obtained by an angle β determined by a distance M from the work operation point W to the carrier lens 4 and a horizonal direction distance N orthogonal to a vertical straight line passing through the center of the carrier lens 4, and a leaning angle α of the carrier lens 4 when the wearer performs an operation.

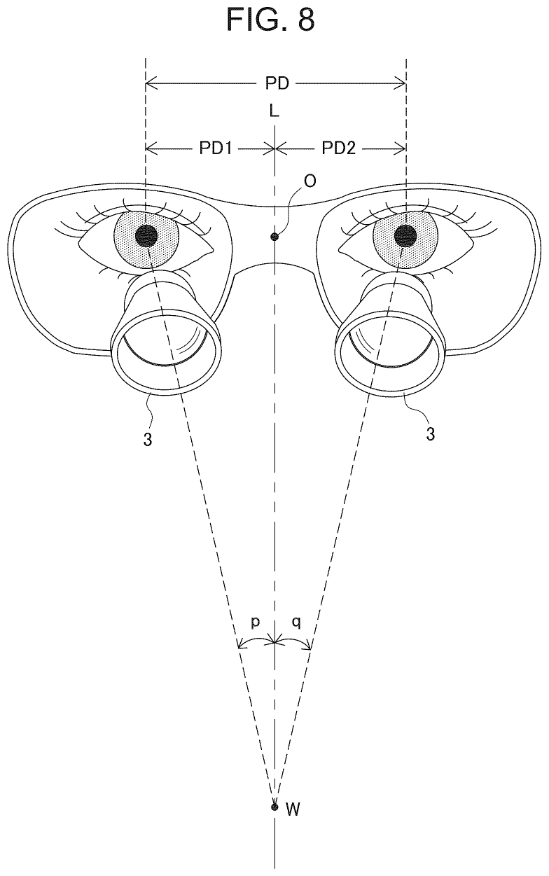

As shown in FIG. 8, the inward insertion angles p, q are angles when lines of sight with the front ends of the right and left loupe bodies 3 inserted into the loupe holders 9 directed toward the operation work point W intersect a line L for joining the center O of the glasses frame 10 and the work operation point W in the work operation point W. Then, the center O is an intersection point of a center line of the nose of the user and a line for connecting between right and left pupils, and the inward insertion angles p, q of the right and left loupe bodies 3 are respectively determined by distances PD1, PD2 from the center O to respective centers of pupils of the right-and-left both eyes, and a distance on the line L from the center O to the work operation point W.

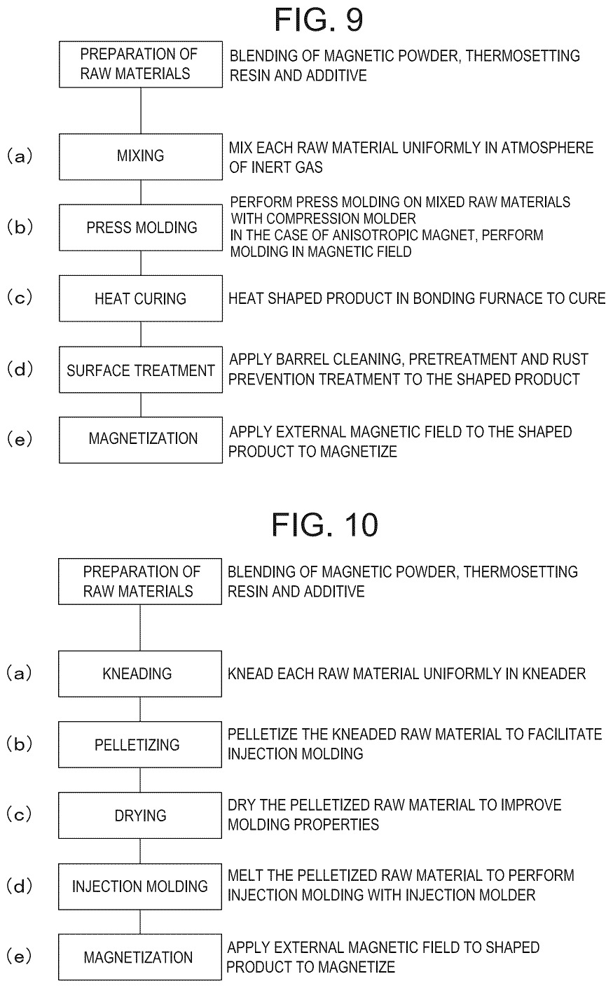

When the downward insertion angle r and inward insertion angles p, q of the loupe bodies 3 are determined, openings to insert the loupe holders 9 are provided in positions corresponding to pupils of both eyes in the right and left carrier lenses 4, the right loupe holder 9 is fixed and attached with an adhesive and the like so as to protrude from the surface of the carrier lens 4 at the downward insertion angle r and inward insertion angle p, and the left loupe holder 9 is fixed and attached with an adhesive and the like so as to protrude from the surface of the carrier lens 4 at the downward insertion angle r and inward insertion angle q. Then, the binocular loupes 100 are used with the loupe body 3 attached to each of the first and second attachment portions of the loupe holder 9, and as necessary, the focus adjustment unit (corresponding to symbol 11 of FIGS. 9 and 10) attached.

As details are described above, the binocular loupes 100 according to the present invention are characterized in that the loupes are provided with a pair of right-and-left loupe bodies 3 with internal optical systems, the glasses frame 10 for holding the carrier lenses 4 to support the pair of loupe bodies 3 in visual directions toward an observation target, and the cylindrical loupe holders 9 attached to the carrier lenses 4 to receive the loupe bodies attachably/detachably with eyepiece side ends of the pair of loupe bodies 3 inserted, and that the loupe holder 9 and eyepiece side members of the pair of loupe bodies 3 are formed of the plastic magnet molded by mixing the magnetic powder into plastic materials.

In addition, the plastic magnet material is capable of obtaining not only products exerting higher magnetic flux densities than those of rubber magnet materials, and of obtaining also extremely thin molded products. The rubber mat materials are soft with flexibility, but are hard to prepare products requiring complicated precision processing.

Further, the plastic magnet material is capable of obtaining not only products comparing favorably with sintered magnet materials in performance of the residual induction, and of obtaining also extremely thin molded products. Furthermore, it is possible to obtain the extremely thin molded product, as mechanically/physically strong products. On the other hand, in the case of manufacturing a thin product using materials by the sintered magnet manufacturing method, the product is brittle, apt to splinter, and high in manufacturing cost.

Therefore, in the present invention, with respect to the contact portions 3a, 3b, 3c on the loupe body 3 side inserted into the cylindrical inner surface of the cylindrical loupe holder 9, all or a part of the portions or more are formed of the plastic magnet member formed by mixing or kneading the plastic material with the magnetic powder.

By this means, each of the pair of loupe bodies 3 is locked closely in the cylindrical inner surface of the loupe holder in an attracted state, by magnetic attraction forces with the contact portions of the loupe bodies 3 in the pair of loupe holders 9. Further, also in the loupe holder 9, all or a part of portions for receiving the contact portions 3a, 3b, 3c on the loupe body 3 side are formed of the plastic magnet member formed by mixing or kneading the plastic material with the magnetic powder.

Thus, the plastic magnet member formed by mixing or kneading the plastic material with the magnetic powder is used to form all or a part of the contact portions 3a, 3b, 3c or more on the loupe body 3 side inserted into the cylindrical inner surface of the cylindrical loupe holder 9, and the portions for receiving the contact portions 3a, 3b, 3c on the loupe body 3 side, and a part of portions formed of the plastic magnet member of the loupe holder 9 and/or the loupe body 3 is magnetized to be an anisotropic plastic magnet described later. By this means, in between the loupe holder 9 and loupe body 3, without receiving magnetic repulsion, conversely, by using the magnetic attraction force and repulsion force smartly, it is also made possible that the loupe body 3 is easily inserted into the barrel of the loupe holder 9.

As such an anisotropic magnetic orientation, there are axial (vertical direction) orientation, radial (radial direction) orientation, repulsion radial orientation, polar anisotropic orientation and the like. In the binocular loupes according to the present invention, the axial orientation is mainly used, but the invention is not limited thereto. An anisotropic orientation by the axial orientation is to extend the oriented magnetic field in the vertical axis direction, and align easy axes of magnetization in the vertical direction to be oriented. By this means, magnets of N/S are made on opposite end surfaces, and it is possible to provide magnetization with strong magnetic attraction force on a one-surface side and/or both-surface sides.

Next, detailed description will be given to a press molding method, injection molding method and the like, which are methods of manufacturing the binocular loupes according to the present invention.

The plastic magnet member used in the press molding method and injection molding method is one of plastic bonded magnets. The plastic bonded magnet means a resin-bonded type magnet obtained by mixing rare earth-based or ferrite magnetic powder with plastic used as a binder to solidify. When necessary, a lubricant and antioxidant are added as additives. Generally, since the plastic bonded magnet contains a resin, as compared with the sintered magnet, although the magnetic flux density meaning the magnetic force is poor slightly, dimension precision is high in the forming finished product, post-processing is thereby eliminated, the mechanical strength is strong, and it is possible to manufacture, at low cost, a thin part and product in a complicated shape required by the binocular loupes.

Then, with respect to performance of the magnetic flux density meaning the magnetic force, by using samarium-iron-nitrogen (SmFeN)-based magnetic powder or neodymium-iron-boron (NdFeB)-based magnetic powder described later, it is made possible that the loupe body 3 constituting the binocular loupes is locked closely in the cylindrical inner surface of the loupe holder 9 in an attracted state, by magnetic attraction forces with contact portions of the loupe body 3 in the loupe holder 9.

Portions using the plastic magnet in preparation of the binocular loupes according to the present invention are at least the cylindrical loupe holder 9 and contact portions 3a, 3b, 3c contacting the cylindrical inner surface of the loupe holder 9 in the loupe body 3, and the entire barrel portion of the loupe holder 9 and the whole or most of the loupe body 3 may be prepared based on preparation methods described below in detail.

FIG. 9 illustrates process steps of a press molding method that is a first manufacturing method of the binocular loupes (cylindrical loupe holders 9 and loupe bodies 3).

In the press molding method that is the first manufacturing method, first, prepared are raw materials obtained by combining the samarium-iron-nitrogen (SmFeN)-based magnetic powder or neodymium-iron-boron (NdFeB)-based magnetic powder described later to be a raw material of the cylindrical loupe holder 9 and loupe body 3, a thermosetting resin, and an additive as necessary.

Then, preparation is made using the above-mentioned raw materials by the following processes a) to e). The processes include that
a) the magnetic powder and thermosetting resin materials are mixed uniformly in an atmosphere of an inert gas,
b) the mixed raw material is subjected to press molding with a compression molder,
c) a shaped product subjected to the press molding is cured in a bonding furnace by heating,
d) the cured shaped product is subjected to barrel cleaning, surface treatment, and then rust prevention treatment, and that
e) the external magnetic field is applied to the shaped product to magnetize.

By this means, in the binocular loupes, since the cylindrical loupe holder 9 and contact portions contacting at least the cylindrical inner surface of the loupe holder in the loupe body 3 are formed using the plastic magnet member obtained by mixing the magnetic powder having strong magnetic properties and mechanical properties with plastic materials, the loupe body 3 is closely locked in the cylindrical inner surface of the loupe holder 9 by magnetic attraction forces, and is thereby capable of being inserted attachably/detachably, without being dropped from the loupe holder.

FIG. 10 illustrates process steps of an injection molding method that is a second manufacturing method of the binocular loupes (cylindrical loupe holders and loupe bodies).

Also in the injection molding method that is the second manufacturing method, prepared are raw materials obtained by combining the samarium-iron-nitrogen (SmFeN)-based magnetic powder or neodymium-iron-boron (NdFeB)-based magnetic powder described later to be a raw material of the cylindrical loupe holder 9 and loupe body 3, a thermoplastic resin, and an additive as necessary, and preparation is made by the following processes a) to e). In other words, the processes include that
a) the above-mentioned magnetic powder and thermoplastic resin materials that are raw materials are charged into a kneader and are uniformly kneaded,
b) the kneaded raw material is pelletized to facilitate injection molding,
c) the pelletized raw material is subjected to dry treatment,
d) the dry-treated pelletized raw material is melted, and the melted raw material is subjected to injection molding into a desired shape die with an injection molder, and that
e) the external magnetic field is applied to the injection-molded shaped product to magnetize.

Next, descriptions will be given to methods of injecting the melted plastic magnet into a shape die in the above-mentioned injection molding method.

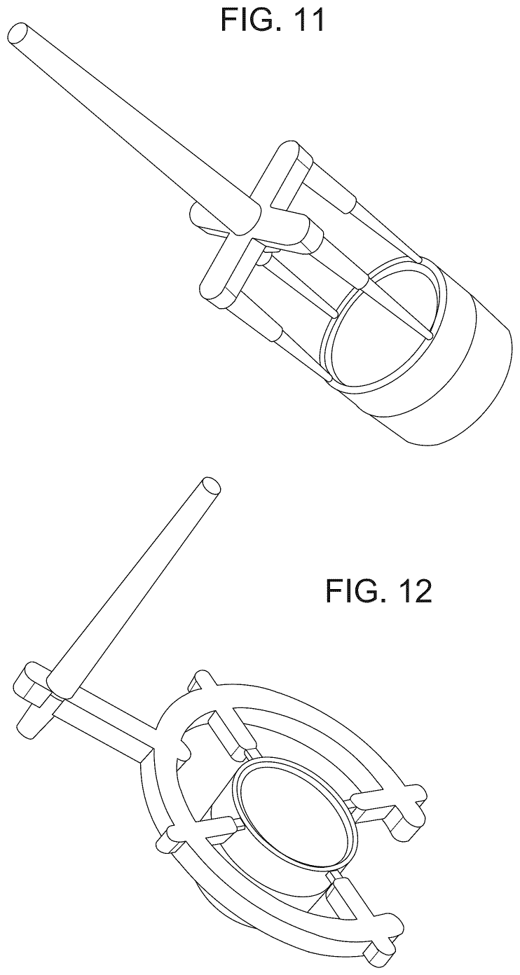

In FIG. 11, the dry-treated pelletized melted raw material is injected into the shape die using a plurality of (“4” in the example of FIG. 11) pinpoint injection gates. By thus injecting into the shape die using a plurality of pinpoint injection gates, even plastic magnet raw materials with relatively high viscosity enter every corner inside the thin cylindrical shape die, and it is possible to mold the plastic magnet in an extremely thin cylindrical shape. The number of pinpoint injection gates (injection exits of the melted plastic magnet) range from about “2” to “4”.

FIG. 12 illustrates an example of injecting the melted raw material (plastic·magnet) into the shape die, using side injection gates that is the injection molding method of the cylindrical loupe holder and so on of the binocular loupes. For example, the side injection gate is suitable for injecting the melted raw material (plastic·magnet) from an arc center portion of an arc-shaped plane.

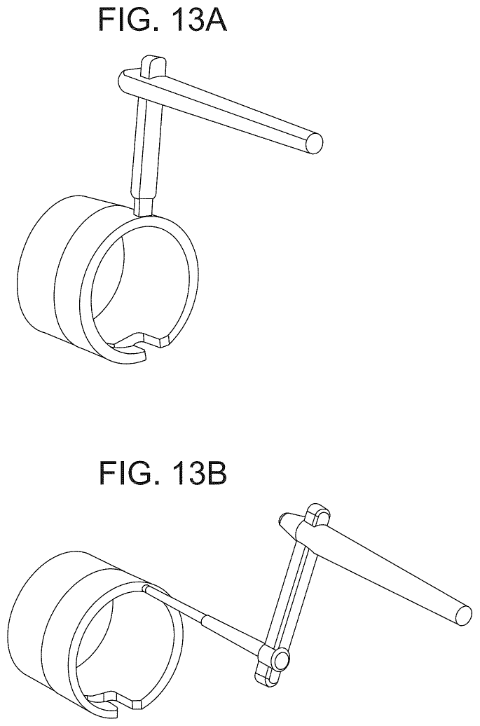

FIGS. 13A and 13B illustrate examples of melted materials using guide injection gates that are the injection molding method of the cylindrical loupe holders and so on of the binocular loupes, FIG. 13A illustrates an example of injecting the melted raw material (plastic·magnet) into a shape die using a guide side injection gate, and FIG. 13B illustrates an example of injecting the melted raw material (plastic·magnet) into the shape die from a guide pin injection gate.

The injection method using the guide side injection gate and injection method using the guide pin injection gate are suitable for the case of injecting the melted plastic magnet into the shape die from the circumference on the side opposite to a notch portion, for example, in the case where (see FIG. 2B, FIGS. 3B and 3C) the notch and like are provided in a part of the cylindrical shape shown in FIG. 13A or FIG. 13B.

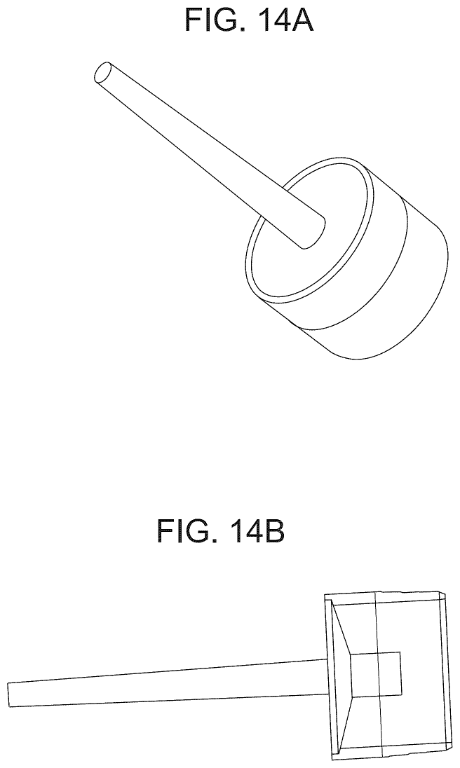

FIGS. 14A and 14B illustrate an example of injecting the melted raw material (plastic·magnet) into a shape die using a disk injection gate that is the injection molding method of the cylindrical loupe holders and so on of the binocular loupes, FIG. 14A illustrates a perspective view thereof, and FIG. 14B illustrates a perspective view thereof viewed from the lateral direction. The injection method using the disk injection gate is suitable for the case of injecting the melted plastic magnet into a shape die to prepare a circular plane-shaped product (for example, vision adjustment lens and astigmatism correction lens).

In the above-mentioned plastic magnet subjected to anisotropic magnetization, since molecules of the raw material are oriented in a certain direction, it is possible to obtain the plastic magnet in a state in which strong magnets limited to one direction are aligned and arranged.

Herein, as the orientation of anisotropy, there are axial (vertical direction) orientation, radial (radial direction) orientation, repulsion radial orientation, polar anisotropic orientation and the like, and in the binocular loupes according to the present invention, the axial orientation is mainly used, but the invention is not limited thereto. The anisotropic orientation by the axial orientation is to extend the oriented magnetic field in the vertical axis direction, and align easy axes of magnetization in the vertical direction to orient. By this means, N/S magnets are made on the opposite end surfaces, and it is possible to provide magnetization with strong magnetic attraction force on a one-surface side and/or both-surface sides.

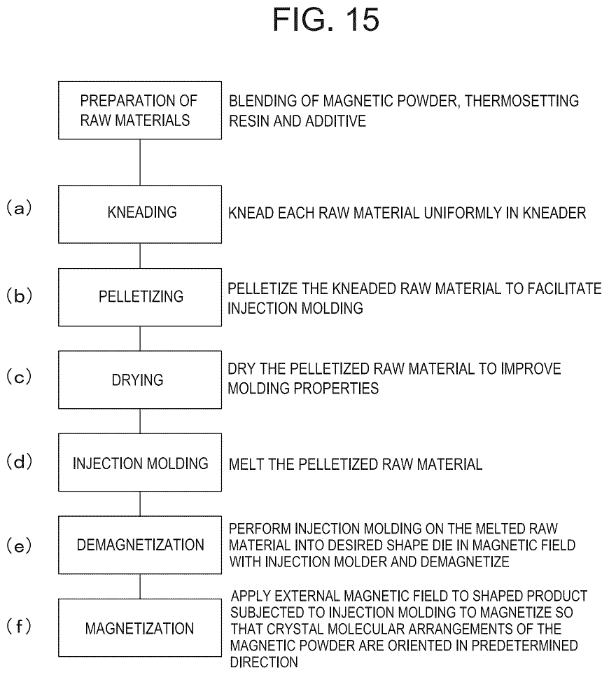

FIG. 15 illustrates processes of generating anisotropy in a magnetic orientation of the magnetic powder of the injection-molded shaped product, in the above-mentioned injection molding method of the binocular loupes (cylindrical loupe holders 9, loupe bodies 3, lens frame of the vision adjustment lens and lens frame of the astigmatism correction lens).

Also in the injection molding method, prepared are raw materials obtained by combining the samarium-iron-nitrogen (SmFeN)-based magnetic powder or neodymium-iron-boron (NdFeB)-based magnetic powder to be the raw material of the cylindrical loupe holder 9, loupe body 3, the lens frame of the vision adjustment lens or the lens frame of the astigmatism correction lens, a thermoplastic resin, and an additive as necessary, and preparation is made by the following processes a) to e). In other words, the processes include that
a) the magnet member constituting the cylindrical loupe holder 9 and at least the contact portions contacting the cylindrical inner surface of the loupe holder in the loupe body 3, and thermoplastic resin materials are charged into a kneader and are uniformly kneaded,
b) the kneaded raw material is pelletized to facilitate injection molding,
c) the pelletized raw material is subjected to dry treatment,
d) the dry-treated pelletized raw material is melted,
e) the melted raw material is subjected to injection molding into a desired shape die in the magnetic field with an injection molder, and is demagnetized as necessary, and that
f) the external magnetic field is applied to the injection-molded shaped product to magnetize so that the crystal molecular arrangement of the magnetic powder is oriented in the predetermined direction.

Next, descriptions will be given to anisotropic magnetization for applying the external magnet field to the injection-molded shaped product to magnetize so that the crystal molecular arrangement of the magnetic powder is oriented in the predetermined direction.

In the binocular loupes according to the present invention, it is necessary to adapt insertion angles (rotation direction angle with reference to the center of the lens surface as the center) with respect to the loupe body in the loupe holder and the vision adjustment lens or the astigmatism correction lens to predetermined angles corresponding to the extent of astigmatism of the practitioner and the like, and therefore, it is required to perform anisotropic magnetization by the above-mentioned process.

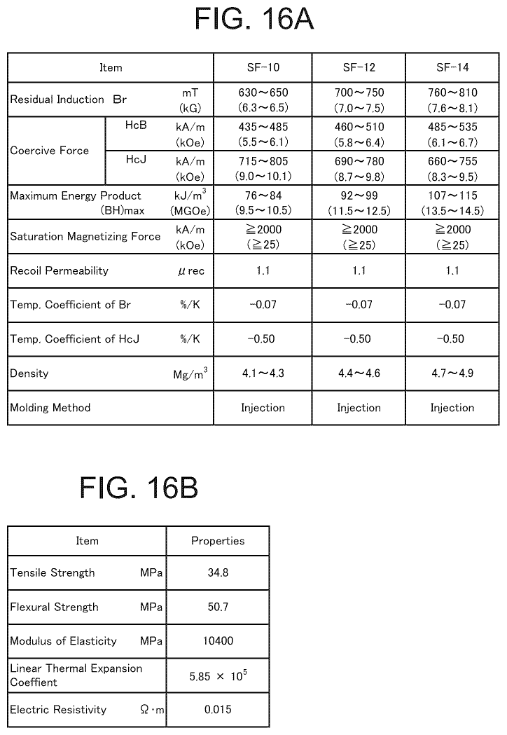

FIGS. 16A and 16B illustrate a product example obtained by mixing or kneading the samarium-iron-nitrogen (SmFeN)-based magnetic powder with plastic materials, as a first example of the plastic magnet member used in preparation of the loupe holders and loupe bodies of the binocular loupes of the present invention, where FIG. 16A shows magnetic properties, and FIG. 16B shows physical mechanical properties.

Herein, the samarium-iron-nitrogen (SmFeN)-based magnetic body is a rare earth magnet developed as a magnet having performance exceeding the neodymium magnet and samarium-cobalt magnet, and enables an anisotropic orientation to be provided. The samarium-iron-nitrogen (SmFeN)-based magnetic body has a magnetic flux density exceeding not only the ferrite-based plastic magnet with an iron oxide as the principal material manufactured by the conventional powder metallurgy method, also the neodymium magnet and samarium-cobalt magnet.

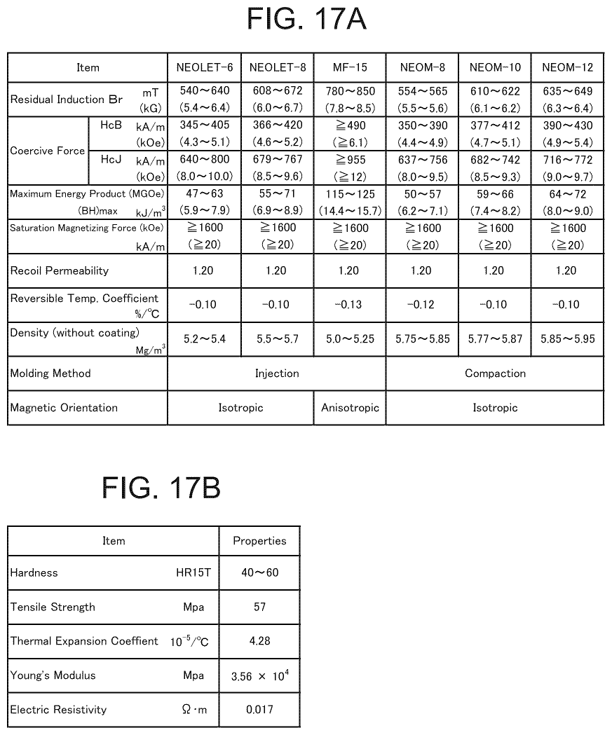

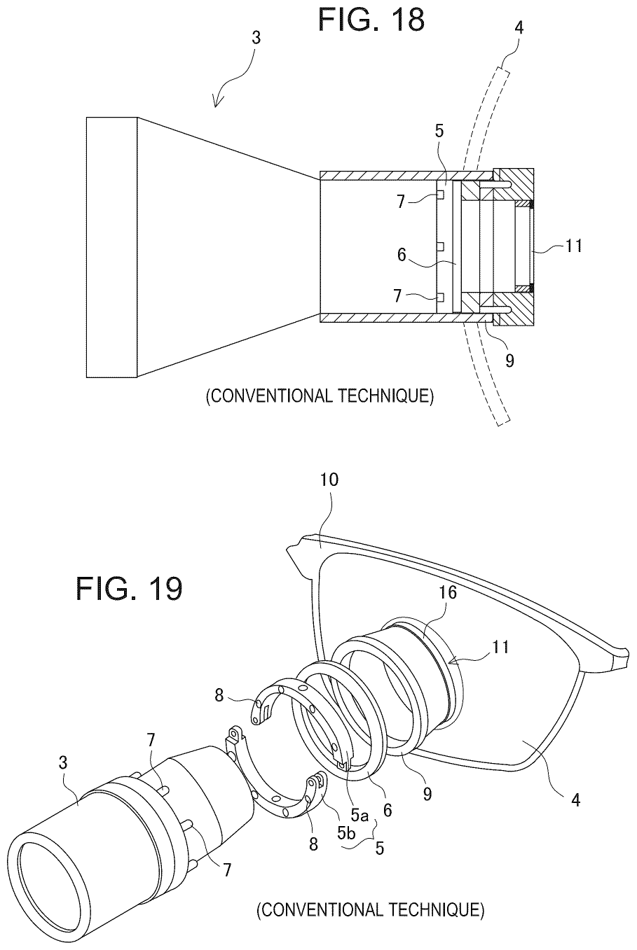

FIGS. 17A and 17B illustrate a product example obtained by mixing or kneading the neodymium-iron-boron (NdFeB)-based magnetic powder with plastic materials, as a second example of the plastic magnet member used in preparation of the loupe holders and loupe bodies of the binocular loupes of the present invention, where FIG. 17A shows magnetic properties, and FIG. 17B shows physical mechanical properties.

Herein, the neodymium-iron-boron (NdFeB)-based magnetic body is a rare earth magnet with Nd (neodymium), Fe (iron) and B (boron) as principal materials, and is a plastic magnet prepared by molding magnetic materials obtained by mixing the NdFeB magnetic powder and epoxy-based thermosetting resin by press molding and the like, and heat curing.

In addition, in FIGS. 16A, 16B, 17A and 17B, the coercive force refers to the strength of the external magnetic force in an opposite direction required to recover the magnetized magnetic body to a state in which the body is not magnetized, and is also called coercivity. In H/B (magnetizing force/magnetic flux density) properties representing properties of a magnetic body, B (magnetic flux density) is indicated when H (magnetizing force) is zero.

As the unit of the coercive force, oersted (Oe) is used in CGS unit system, ampere per meter (A/m) is used in SI unit, and 1 (A/m) is 4n×10³ (Oe).

In addition, as shown in FIG. 6, the loupe body 3 is inserted into the loupe holder 9 via the protrusion-provided ring 12 having the protrusion portion 12a, the loupe holder 9 has the engagement portion 9b of the hook-shaped groove to engage in the protrusion portion 12a, and the loupe body 3 is formed not to be removed from the loupe holder 9.

Herein, the protrusion-provided ring 12 is formed of the plastic magnet molded by mixing the magnetic powder into plastic materials.

Further, the binocular loupes are characterized in that at least each of the loupe holder 9 and loupe body 3 is formed of the anisotropic magnetized material where crystal molecular arrangements of the magnetic powder are oriented in the certain direction, and that each of the loupe bodies 3 is inserted in a predetermined rotation direction with respect to the loupe holder 9.

As described above, the binocular loupes according to the present invention are provided with a pair of right-and-left loupe bodies 3 with internal optical systems, the glasses frame 10 to hold the carrier lenses 4 for supporting the pair of loupe bodies 3 in visual directions toward an observation target, and the cylindrical loupe holders 9 attached to the carrier lenses 4 to receive the loupe bodies 3 attachably/detachably with eyepiece side ends of the pair of loupe bodies 3 inserted, the loupe holders 9 and eyepiece side members of the pair of loupe bodies 3 are formed to be thin and high in magnetic flux density performance, by the method of manufacturing the plastic magnet material molded by mixing the magnetic powder into plastic materials by precision Europe high compression molding, multi-gate injection molding or the like.

By this means, in the binocular loupes 100, the loupe body 3 is closely locked in the cylindrical inner surface of the loupe holder 9 by magnetic attraction forces, is thereby not dropped from the loupe holder 9, and as described later, enables the practitioner to select arbitrary one from a plurality of loupe bodies to easily insert and replace.

Then, in the binocular loupes enabling loupe bodies provided with internal optical systems for enlarging an observation target to visually identify and focus adjustment units to be replaced with loupe bodies 3 having a desired magnification, or with loupe bodies 3 having zoom mechanisms capable of varying continuously the focal distance in a certain range, as compared with the binocular loupes of conventional techniques, it is made possible to provide the binocular loupes provided with brighter clear image quality, while widening the field of view of a practitioner, and to provide the methods of manufacturing the binocular loupes.

### DESCRIPTION OF SYMBOLS

3 Loupe body
4 Carrier lens
9 Loupe holder
9b Engagement portion of the loupe holder
10 Glasses frame
12 Protrusion-provided ring
12a Protrusion portion
100 Binocular loupes

1. Binocular loupes for medical practitioners, comprising:
a pair of right-and-left loupe bodies provided with internal optical lens systems adapted to enlarge an observation target on hand;
right-and-left carrier lenses adapted to support the pair of loupe bodies in visual directions toward the observation target;
a glasses frame adapted to hold the carrier lenses; and
a pair of cylindrical loupe holders adapted to attach the pair of loupe bodies respectively to the right-and-left carrier lenses attachably/detachably, wherein the cylindrical loupe holders and contact portions contacting cylindrical inner surfaces of the loupe holders in the loupe bodies are formed of a plastic magnet member formed by mixing or kneading a predetermined plastic material with predetermined magnetic powder,
the pair of loupe bodies are respectively locked closely in states of being attracted in the cylindrical inner surfaces of the loupe holders by magnetic attraction forces with the contact portions of the loupe bodies in the pair of loupe holders, and
a lubricant is applied to contact surfaces hitting cylindrical front end surfaces of the loupe holders to come into contact and eyepiece end side surfaces of the loupe bodies to improve slidability therebetween.

2. The binocular loupes for medical practitioners according to claim 1, wherein the loupe bodies are inserted into the loupe holders further through rings having protrusion portions, the loupe holders include engagement portions of hook-shaped grooves to engage in the protrusion portions, and the loupe bodies are formed not to be removed from the loupe holders.

3. The binocular loupes for medical practitioners according to claim 1, wherein in the loupe bodies, a plurality of kinds of loupe bodies is beforehand prepared where the loupe bodies are provided with zoom mechanisms capable of varying a focal distance continuously in a single magnification with different focal distances or in a predetermined range, and one is selected from among the bodies to attach to respective one of the loupe holders.

4. The binocular loupes for medical practitioners according to claim 3, wherein one of beforehand prepared vision adjustment lenses for far-sightedness and near-sightedness and astigmatism correction lenses is selected and fitted into respective one of the eyepiece end side surfaces of the loupe bodies attachably/detachably.

5. A method of manufacturing the binocular loupes for medical practitioners according to claim 1,
wherein the plastic magnet member constituting the cylindrical loupe holders and at least the contact portions contacting the cylindrical inner surfaces of the loupe holders in the loupe bodies is prepared by processes including:
a) mixing the magnetic powder with a thermosetting resin material uniformly in an atmosphere of an inert gas;
b) performing press molding on mixed raw materials with a compression molder;
c) curing a shaped product subjected to the press molding in a bonding furnace by heating;
d) applying barrel cleaning, surface treatment, and then, rust prevention treatment to the shaped product cured; and
e) applying an external magnetic field to the shaped product to magnetize.

6. A method of manufacturing the binocular loupes for medical practitioners according to claim 1,
wherein the plastic magnet member constituting the cylindrical loupe holders and at least the contact portions contacting the cylindrical inner surfaces of the loupe holders in the loupe bodies is prepared by processes including:
a) charging the magnetic powder and a thermoplastic resin material into a kneader to be uniformly kneaded;
b) pelletizing kneaded raw materials to facilitate injection molding;
c) applying dry treatment to the raw materials subjected to the pelletizing;
d) melting the raw materials subjected to the pelletizing and the dry treatment, performing injection molding on melted raw materials into a desired shape die with an injection molder; and
e) applying an external magnetic field to a shaped product subjected to the injection molding to magnetize.

7. The method of manufacturing the binocular loupes for medical practitioners according to claim 6, wherein in process d), the injection molder injects the melted raw materials into the shape die from one or a plurality of pinpoint injection gates.

8. The method of manufacturing the binocular loupes for medical practitioners according to claim 6, wherein in process d), the injection molder injects the melted raw materials into the shape die using one or a plurality of side injection gates.

9. The method of manufacturing the binocular loupes for medical practitioners according to claim 6, wherein in process d), the injection molder injects the melted raw materials into the shape die using a disk injection gate.

10. The binocular loupes for medical practitioners according to claim 1, wherein in order for each of the pair of loupe bodies to be inserted in a predetermined rotation direction with respect to the loupe holders, at least each of the loupe holders and loupe bodies is formed of an anisotropic magnetized material where a crystal molecular arrangement of the magnetic powder is oriented in a predetermined direction.

11. A method of manufacturing the binocular loupes for medical practitioners according to claim 10,
wherein the plastic magnet member constituting the cylindrical loupe holders and at least the contact portions contacting the cylindrical inner surfaces of the loupe holders in the loupe bodies is prepared by processes including:
a) charging the magnetic powder and a thermoplastic resin material into a kneader to be uniformly kneaded;
b) pelletizing kneaded raw materials to facilitate injection molding;
c) allying dry treatment to the raw materials subjected to the pelletizing;
d) melting the raw materials subjected to the pelletizing and the dry treatment;
e) performing injection molding on the raw materials melted into a desired shape die in a magnetic field with an injection molder, further demagnetizing as necessary; and
f) applying an external magnetic field to a shaped product subjected to the injection molding to magnetize so that a crystal molecular arrangement of the magnetic powder is oriented in a predetermined direction.

12. The method of manufacturing the binocular loupes for medical practitioners according to claim 5, wherein the plastic magnet member is a structural member obtained by mixing or kneading samarium-iron-nitrogen (SmFeN)-based magnetic powder with a predetermined plastic material.

13. The method of manufacturing the binocular loupes for medical practitioners according to claim 12, wherein the plastic magnet member is a structural member obtained by mixing or kneading samarium-iron-nitrogen (SmFeN)-based magnetic powder containing a ferrite iron oxide with a predetermined plastic material.

14. The method of manufacturing the binocular loupes for medical practitioners according to claim 5, wherein the plastic magnet member is a structural member obtained by mixing or kneading neodymium-iron-boron (NdFeB)-based magnetic powder with a predetermined plastic material.

15. The method of manufacturing the binocular loupes for medical practitioners according to claim 5, wherein the processes further including:
f) fixing the loupe holders at predetermined angles with respect to surfaces of the loupe holders so that the loupe bodies inserted are directed toward the observation target.

16. The method of manufacturing the binocular loupes for medical practitioners according to claim 6, wherein the processes further including:
g) fixing the loupe holders at predetermined angles with respect to surfaces of the loupe holders so that the loupe bodies inserted are directed toward the observation target.

17. The method of manufacturing the binocular loupes for medical practitioners according to claim 15, wherein in the loupe bodies, a plurality of kinds of loupe bodies is beforehand prepared where the loupe bodies are provided with zoom mechanisms capable of varying a focal distance continuously in a single magnification with different focal distances or in a predetermined range, and one is selected from among the bodies to attach to respective one of the loupe holders.

18. The method of manufacturing the binocular loupes for medical practitioners according to claim 17, wherein one of beforehand prepared vision adjustment lenses for far-sightedness and near-sightedness and astigmatism correction lenses is selected and fitted into respective one of the eyepiece end side surfaces of the loupe bodies attachably/detachably.

19. The method of manufacturing the binocular loupes for medical practitioners according to claim 18, wherein the vision adjustment lenses or the astigmatism correction lenses are held by rims surrounding circumferences of the lenses, and the rims are formed of a ferrite magnetic body.

20. The method of manufacturing the binocular loupes for medical practitioners according to claim 19, wherein the plastic magnet member constituting the vision adjustment lenses or the astigmatism correction lenses is prepared by processes including:
a) charging the magnetic powder and a thermoplastic resin material into a kneader to be uniformly kneaded;
b) pelletizing kneaded raw materials to facilitate injection molding;
c) applying dry treatment to the raw materials subjected to the pelletizing;
d) melting the raw materials subjected to the pelletizing and the dry treatment;
e) performing injection molding on the raw materials melted into a desired shape die in a magnetic field with an injection molder, further demagnetizing as necessary; and
f) applying an external magnetic field to a shaped product subjected to the injection molding to magnetize so that a crystal molecular arrangement of the magnetic powder is oriented in a predetermined direction.

21. The method of manufacturing the binocular loupes for medical practitioners according to claim 19, wherein the plastic magnet member constituting the vision adjustment lenses or the astigmatism correction lenses is a structural member obtained by mixing or kneading samarium-iron-nitrogen (SmFeN)-based magnetic powder with a predetermined plastic material.

22. The method of manufacturing the binocular loupes for medical practitioners according to claim 19, wherein the plastic magnet member constituting the vision adjustment lenses or the astigmatism correction lenses is a structural member obtained by mixing or kneading samarium-iron-nitrogen (SmFeN)-based magnetic powder containing a ferrite iron oxide with a predetermined plastic material.

23. The method of manufacturing the binocular loupes for medical practitioners according to claim 17, wherein the plastic magnet member constituting the vision adjustment lenses or the astigmatism correction lenses is a structural member obtained by mixing or kneading neodymium-iron-boron (NdFeB)-based magnetic powder with a predetermined plastic material.
